(19) United States
(12) Patent Application Publication
(10) Pub. No.: US 2021/0196501 A1
(43) Pub. Date: Jul. 1, 2021

(54) Male External Catheterization System

(71) Applicant: Jocelyn Sison, Lake Villa, IL (US)

(72) Inventor: Jocelyn Sison, Lake Villa, IL (US)

(21) Appl. No.: 16/728,726

(22) Filed: Dec. 27, 2019

(51) Int. Cl. A61F 5/453; A61F 5/443

(52) U.S. Cl. CPC A61F 5/453; A61F 5/443

(57) ABSTRACT

A male external catheterization system for a nonmobile male user includes a panel, which is pliable and has an orifice substantially centrally positioned therethrough. A tube is coupled to an outer face of the panel and extends from the orifice. The tube is circumferentially larger proximate to the panel. At least a portion of a penis is insertable into the tube from an inner face of the panel. The tube is engaged a suction unit that suctions urine that exits the penis through the tube into the suction unit.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to catheterization systems and more particularly pertains to a new catheterization system for a nonmobile male user.

### (2) Description of Related Art Including Information Disclosed Under 37 CFR 1.97 and 1.98

The prior art relates to catheterization systems. Prior art external catheter devices may comprise a receptacle for inserting either genitalia or a penis of a user and a tube connecting the receptacle to a gravity fed reservoir that may contain an absorbent material.

### BRIEF SUMMARY OF THE INVENTION

An embodiment of the disclosure meets the needs presented above by generally comprising a panel, which is pliable and has an orifice substantially centrally positioned therethrough. A tube is coupled to an outer face of the panel and extends from the orifice. The tube is circumferentially larger proximate to the panel and is configured for insertion of at least a portion of a penis from an inner face of the panel. The tube is configured to engage a suction unit, which is configured to suction urine that exits the penis through the tube into the suction unit.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a male external catheterization system according to an embodiment of the disclosure.

FIG. 2 is a front view of an embodiment of the disclosure.

FIG. 3 is a rear view of an embodiment of the disclosure.

FIG. 4 is a side view of an embodiment of the disclosure.

FIG. 5 is an in-use view of an embodiment of the disclosure.

### DETAILED DESCRIPTION OF THE INVENTION

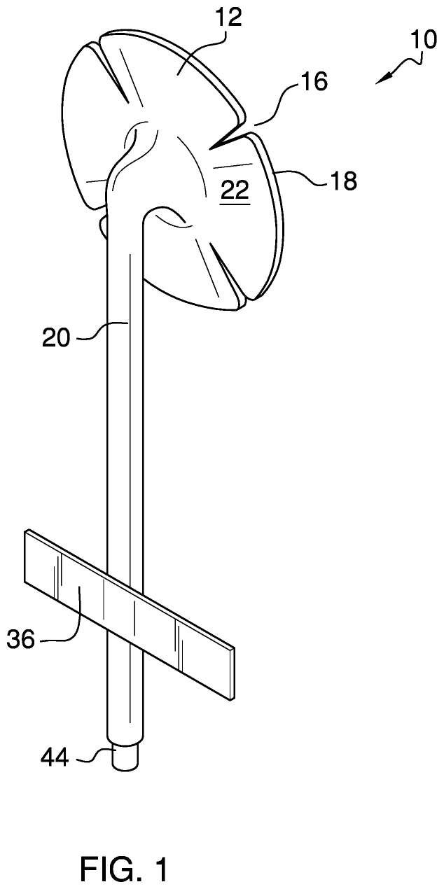

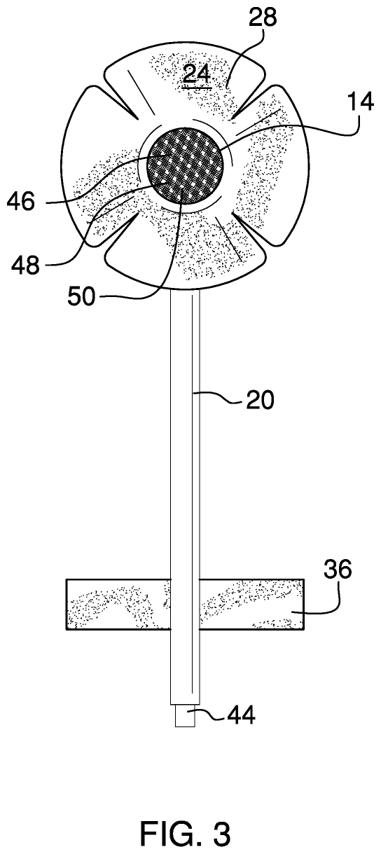

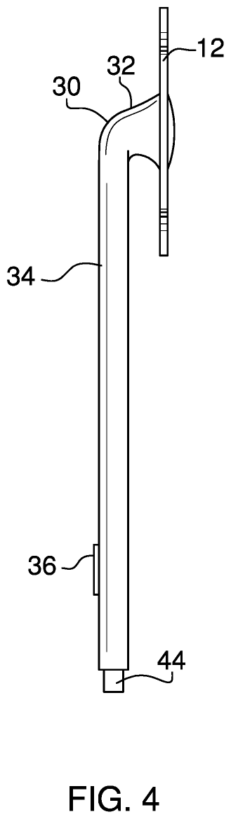

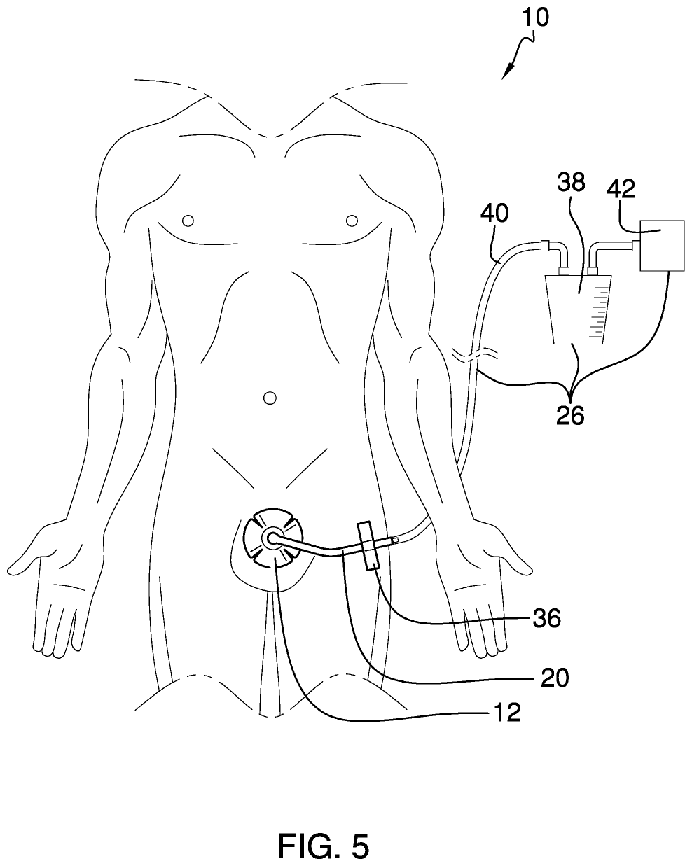

With reference now to the drawings, and in particular to FIGS. 1 through 5 thereof, a new catheterization system embodying the principles and concepts of an embodiment of the disclosure and generally designated by the reference numeral 10 will be described.

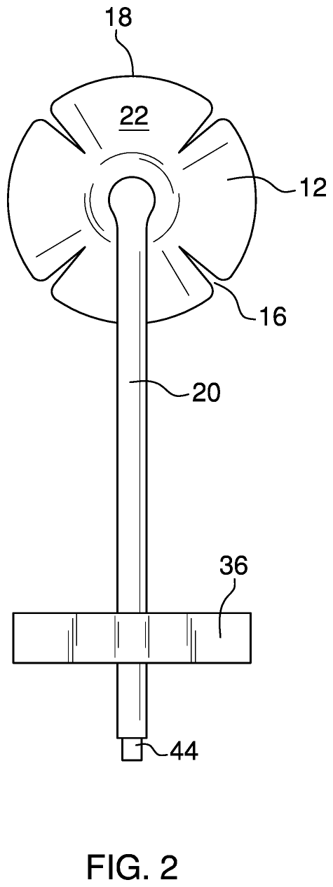

As best illustrated in FIGS. 1 through 5, the male external catheterization system 10 generally comprises a panel 12, which is pliable and has an orifice 14 substantially centrally positioned therethrough. The panel 12 may be substantially circularly shaped, as shown in FIG. 2, or alternatively shaped, such as ovally shaped, rectangularly shaped, and the like.

The panel 12 has a cutout 16 positioned therein which extends into the panel 12 from a perimeter 18 of the panel 12. As will become apparent, the cutout 16 allows the panel 12 to conform to contours of a pelvic region of a user. The cutout 16 is V-shaped so that the cutout 16 is dimensionally narrower distal from the perimeter 18. The cutout 16 being V-shaped allows for the cutout 16 to contract and helps in conforming the panel 12 to the contours of the pelvic region of the user. The panel 12 may comprise silicone, or other pliable material, such as, but not limited to, rubber, elastomer, and the like.

A tube 20 is coupled to an outer face 22 of the panel 12 and extends from the orifice 14. The tube 20 is circumferentially larger proximate to the panel 12 and is configured for insertion of at least a portion of a penis from an inner face 24 of the panel 12. The tube 20 is configured to engage a suction unit 26, which is configured to suction urine that exits the penis through the tube 20 into the suction unit 26. An adhesive 28 is engaged to the inner face 24 of the panel 12 and is configured to adhesively couple the panel 12 to skin proximate to the penis.

The tube 20 has a bend 30 positioned therein proximate to the panel 12. The bend 30 defines a first segment 32 and a second segment 34 of the tube 20. The first segment 32 is circumferentially larger than the second segment 34. The first segment 32 is configured for insertion of the at least the portion of the penis. The bend 30 is positioned so that the second segment 34 is substantially parallel to the panel 12. This positioning of the bend 30 keeps the tube 20 proximate to the user.

The first segment 32 tapers from the panel 12 to the second segment 34 so that the first segment 32 is circumferentially larger proximate to the panel 12. The first segment 32 thus is configured to sealably couple to the penis when a partial vacuum is applied by the suction unit 26.

A fastener 36 is engaged to the tube 20 distal from the panel 12 and is configured to selectively engage skin of the user to fasten the tube 20 to the user. The fastener 36 may be adhesive tape type, to fasten the tube 20 to skin, or other fastening type to fasten the tube 20 to an article of clothing or a surface proximate to the user, such as clip type, hook and loop type, and the like.

The suction unit 26 comprises a reservoir 38, a conduit 40, and a suctioning device 42. The conduit 40 is engaged to and extends from the reservoir 38 and is configured to engage the tube 20 to fluidically couple the tube 20 to the reservoir 38. The suctioning device 42 is engaged to the reservoir 38 and is configured to partially evacuate air from the reservoir 38 to suction the urine that exits the penis through the tube 20 and the conduit 40 into the reservoir 38. A connector 44 is engaged to the tube 20 distal from the panel 12 and is configured to engage the conduit 40 to fluidically couple the tube 20 to the reservoir 38.

A first sheath 46 is engaged to an inner surface 48 of the first segment 32 of the tube 20 and is configured to wick urine from the first segment 32 into the second segment 34. A second sheath 50 is engaged to the first sheath 46 and is configured to transfer the urine to the first sheath 46. The second sheath 50 serves to separate the penis from the urine in the first sheath 46. Together, the first sheath 46 and the second sheath 50 function to keep the penis dry.

In use, the penis of the user is inserted into the tube 20 and the panel 12 is pressed against the skin so that the adhesive 28 engages the skin to couple the panel 12 to the user. The tube 20 is connected to the conduit 40 using the connector 44, as which time the suctioning device 42 can be actuated to partially evacuate the reservoir 38. When the user urinates, the urine passes through the tube 20 and the conduit 40 to the reservoir 38.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context clearly requires that there be only one of the elements.

I claim:

1. A male external catheterization system comprising:
a panel, the panel being pliable, the panel having an orifice substantially centrally positioned therethrough; and
a tube coupled to an outer face of the panel and extending from the orifice, the tube being circumferentially larger proximate to the panel, wherein the tube is configured for insertion of at least a portion of a penis from an inner face of the panel, the tube being configured for engaging a suction unit, wherein the suction unit is configured for suctioning urine exiting the penis through the tube into the suction unit.

2. The male external catheterization system of claim 1, wherein the panel is substantially circularly shaped.

3. The male external catheterization system of claim 1, further including the panel having a cutout positioned therein, the cutout extending into the panel from a perimeter of the panel.

4. The male external catheterization system of claim 3, wherein the cutout is V-shaped such that the cutout is dimensionally narrower distal from the perimeter.

5. The male external catheterization system of claim 1, wherein the panel comprises silicone.

6. The male external catheterization system of claim 1, further including the tube having a bend positioned therein proximate to the panel defining a first segment and a second segment of the tube, the first segment being circumferentially larger than the second segment, wherein the first segment is configured for insertion of the at least the portion of the penis.

7. The male external catheterization system of claim 6, wherein the bend is positioned such that the second segment is substantially parallel to the panel.

8. The male external catheterization system of claim 6, wherein the first segment tapers from the panel to the second segment, such that the first segment is circumferentially larger proximate to the panel wherein the first segment is configured for sealably coupling to the penis.

9. The male external catheterization system of claim 1, further including the suction unit comprising:
a reservoir;
a conduit engaged to and extending from the reservoir and being configured for engaging to the tube for fluidically coupling the tube to the reservoir; and
a suctioning device engaged to the reservoir and being configured for partially evacuating air from the reservoir for suctioning the urine exiting the penis through the tube and the conduit into the reservoir.

10. The male external catheterization system of claim 9, further including a connector engaged to the tube distal from the panel and being configured for engaging the conduit for fluidically coupling the tube to the reservoir.

11. The male external catheterization system of claim 6, further including a first sheath engaged to an inner surface of the first segment of the tube and being configured for wicking urine from the first segment into the second segment.

12. The male external catheterization system of claim 11, further including a second sheath engaged to the first sheath and being configured for transferring the urine to the first sheath.

13. The male external catheterization system of claim 1, further including a fastener engaged to the tube distal from the panel and being configured for selectively engaging skin of a user for fastening the tube to the user.

14. The male external catheterization system of claim 13, wherein the fastener is adhesive tape type.

15. The male external catheterization system of claim 1, further including an adhesive engaged to the inner face of the panel and being configured for adhesively coupling the panel to skin proximate to the penis.

16. A male external catheterization system comprising:
a panel, the panel being pliable, the panel having an orifice substantially centrally positioned therethrough, the panel being substantially circularly shaped, the panel having a cutout positioned therein, the cutout extending into the panel from a perimeter of the panel, the cutout being V-shaped such that the cutout is dimensionally narrower distal from the perimeter, the panel comprising silicone;
a tube coupled to an outer face of the panel and extending from the orifice, the tube being circumferentially larger proximate to the panel, wherein the tube is configured for insertion of at least a portion of a penis from an inner face of the panel, the tube being configured for engaging a suction unit, wherein the suction unit is configured for suctioning urine exiting the penis through the tube into the suction unit, the tube having a bend positioned therein proximate to the panel defining a first segment and a second segment of the tube, the first segment being circumferentially larger than the second segment, wherein the first segment is configured for insertion of the at least the portion of the penis, the bend being positioned such that the second segment is substantially parallel to the panel, the first segment tapering from the panel to the second segment, such that the first segment is circumferentially larger proximate to the panel wherein the first segment is configured for sealably coupling to the penis;
the suction unit comprising:
a reservoir,
a conduit engaged to and extending from the reservoir and being configured for engaging to the tube for fluidically coupling the tube to the reservoir, and
a suctioning device engaged to the reservoir and being configured for partially evacuating air from the reservoir for suctioning the urine exiting the penis through the tube and the conduit into the reservoir;
a connector engaged to the tube distal from the panel and being configured for engaging the conduit for fluidically coupling the tube to the reservoir;
a first sheath engaged to an inner surface of the first segment of the tube and being configured for wicking urine from the first segment into the second segment;
a second sheath engaged to the first sheath and being configured for transferring the urine to the first sheath;
a fastener engaged to the tube distal from the panel and being configured for selectively engaging skin of a user for fastening the tube to the user, the fastener being adhesive tape type; and
an adhesive engaged to the inner face of the panel and being configured for adhesively coupling the panel to skin proximate to the penis.
